(19) United States
(12) Patent Application Publication
(10) Pub. No.: US 2024/0351971 A1
(43) Pub. Date: Oct. 24, 2024

(54) PROCESS FOR EXTRACTING FERULIC ACID AND/OR SALTS THEREOF, COMPRISING A STEP A) IN WHICH A BIOMASS IS EXTRUDED IN THE PRESENCE OF A BASE

(71) Applicant: SPECIALTY OPERATIONS FRANCE, Lyon (FR)

(72) Inventor: Rodolphe Sapey-Triomphe, Corbas (FR)

(73) Assignee: SPECIALTY OPERATIONS FRANCE, Lyon (FR)

(21) Appl. No.: 18/291,734

(22) PCT Filed: Jul. 22, 2022
(86) PCT No.: PCT/EP2022/071021

§ 371 Date: Jan. 24, 2024

(30) Foreign Application Priority Data
Jul. 27, 2021 (FR) FR2108154

(51) Int. Cl. C07C 51/47; C07C 51/43; C12P 7/24

(52) U.S. Cl. CPC C07C 51/47; C07C 51/43; C12P 7/24

(57) ABSTRACT

The present invention relates to a method for extracting ferulic acid and/or its salts, comprising a step (a) in which a biomass is extruded in the presence of a base. The present invention also relates to ferulic acid in protonated or salified form that can be obtained according to the method of the present invention. Finally, the present invention also relates to a method for preparing vanillin from ferulic acid obtained according to the method of the present invention. The invention can be used in particular in the field of food, cosmetics and flavours.

### BRIEF DESCRIPTION

The present invention relates to a process for extracting ferulic acid and/or salts thereof, comprising a step (a) in which a biomass is extruded in the presence of a base. The present invention also relates to ferulic acid in protonated or salified form that may be obtained according to the process of the present invention. Finally, the present invention also relates to a process for preparing vanillin from ferulic acid obtained according to the process of the present invention.

The invention finds applications notably in the field of foodstuffs, cosmetics and flavorings.

### PRIOR ART

Ferulic acid, or 3-(4-hydroxy-3-methoxyphenyl) prop-2-enoic acid, is a compound naturally present in plants, notably cereals such as rice, corn, wheat and oats. It may also be present in solid or liquid coproducts from the agrifood industry, in particular the oilseed, cereal, sugar and alcohol industries.

Ferulic acid may be prepared by chemical synthesis, or via a biotechnological route involving microbial fermentation or plant tissue culture. It may also be obtained via a route qualified as natural and/or biobased, in which plant material is processed so as to extract ferulic acid from said plant material. For example, it may be extracted from byproducts of the agrifood industry or from grains, for example according to the process described in WO 2014/187784.

WO 2001/067891 describes a preparation process for separating ferulic acid and arabinoxylans, comprising an extrusion step followed by a step in which the extrudate is suspended in water in the presence of enzymes enabling hydrolysis of the cell walls. Table 1 moreover appears to indicate that an extrusion process alone does not allow the separation of ferulic acid from rice bran.

Ferulic acid is used in various fields ranging from cosmetics to foodstuffs, in particular in the preparation of a widely consumed flavoring substance, vanillin.

Vanillin may be produced by chemical synthesis, but consumers prefer natural flavorings to synthetic ones. In order to meet current demand, particular interest has been paid to the preparation of non-synthetic vanillin. Thus, methods for preparing natural vanillin using natural and/or biobased materials have been developed, these methods being permitted to be qualified as natural under current legislation.

In particular, natural vanilla may be obtained via a biotechnological process notably comprising the cultivation of a microorganism that is capable of achieving the biotransformation of a fermentation substrate into vanillin. Such a biotechnological process is described, for example, in patent application EP 0885968, in which a microorganism converts ferulic acid into vanillin. The natural vanillin thus obtained generally undergoes extraction and/or purification steps. For example, vanillin may be purified according to the methods described in WO 2014/114590, EP 2791098 or WO 2018/146210.

### DESCRIPTION OF THE FIGURES

FIG. 1 is a block diagram describing a first embodiment of the process for extracting ferulic acid and/or salts thereof according to the present invention.

FIG. 2 is a block diagram describing a second embodiment of the process for extracting ferulic acid and/or salts thereof according to the present invention.

FIG. 3 shows the results obtained in Example 8.

### DETAILED DESCRIPTION

In the context of the present invention, and unless otherwise indicated, the term “comprising . . . ” also includes the meaning of “consisting of . . . ”.

In the context of the present invention, and unless otherwise indicated, the expression “between x and y” includes the values x and y. In the context of the present invention, and unless otherwise indicated, the term “ppm” means “parts per million”. This unit represents a mass fraction: 1 ppm=1 mg/kg.

Ferulic acid corresponds to the following formula:

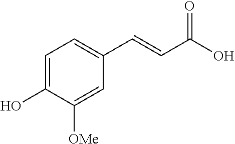 [SMILES: COc1cc(/C=C/C(=O)O)ccc1O]

In the context of the present invention, the expression “biobased ferulic acid” refers to ferulic acid entirely or significantly of plant or marine origin. For example, biobased ferulic acid may be derived from agricultural byproducts, plants, seeds, forestry materials or algae. In particular, biobased ferulic acid is of plant origin. Thus, biobased ferulic acid is not derived from chemical synthesis. In the context of the present invention, the carbon-carbon double bond of biobased ferulic acid is in the trans configuration.

### Step a)

The present invention relates to a process for extracting ferulic acid and/or salts thereof, comprising a step a) in which a biomass is extruded in the presence of a base. The biomass is subjected to reactive extrusion. Step a) allows the preparation of a raw extrudate (EB) and optionally a filtrate (F).

The amount of raw extrudate (EB) is equal to the sum of the masses of the raw materials entering step a); for example, in one particular embodiment, the amount of raw extrudate (EB) may be equal to the mass of the starting biomass plus the mass of base used.

In the context of the present invention, the biomass may be chosen from the group consisting of plants, seeds, forestry materials, algae or agricultural byproducts. In particular, the biomass may be chosen from the group consisting of plant fibers or plant bran, in particular chosen from the group consisting of oat, barley, wheat, rice or corn fibers or bran.

In general, the ferulic acid content in the biomass to be treated is between 0.3% by weight and 5% by weight, preferably between 0.5% and 5% by weight. The ferulic acid content is expressed by weight relative to the weight of the dry biomass.

Step a) is an extrusion step, in particular extractive extrusion or reactive extrusion. In particular, the extrusion may be performed in a corotating twin-screw extruder. The corotating twin-screw extruder may be of the Evolum range from Clextral or Leistritz ZSE. The screw profile may comprise at least one conveying zone, at least one blending zone and possibly counter-threads.

According to a particular aspect, step a) is performed at a temperature of less than or equal to 120° C., preferably less than or equal to 110° C., and very preferentially less than or equal to 100° C. In general, step a) is performed at a temperature greater than or equal to room temperature, preferably greater than or equal to 30° C., very preferentially greater than or equal to 50° C. According to a particular aspect, step a) may be performed at a temperature of between 60° C. and 80° C. In the context of the present invention, room temperature refers to a temperature between 15° C. and 25° C.

The rotation speed of the extruder screws is not particularly limited so as to allow good separation of the ferulic acid contained in the biomass. Said speed may be adapted by a person skilled in the art, taking into account his or her knowledge and notably the type of extruder chosen, the size of the extruder, the screw profile and/or the biomass feed rate. The extruder rotation speed refers to the rotation speed of the extruder screws.

By way of illustration, step a) may be performed with an extruder rotation speed of between 100 rpm and 1200 rpm.

In general, step a) is performed at an extruder rotation speed of between 150 rpm and 400 rpm, for example at 200 rpm.

Thus, as mentioned previously, step a) is performed in the presence of a base. Thus, the biomass is subjected to extraction of ferulic acid and/or salts thereof using a base, simultaneously with extrusion.

The biomass used in the context of the present invention may be wet or dry. According to one embodiment, the biomass is a wet biomass, preferably with a dry matter content of between 35% and 50% by weight, preferably between 38% and 45% by weight. According to another embodiment, the biomass is a dry biomass, preferably with a dry matter content of between 80% and 95% by weight, preferably between 84% and 92% by weight.

In general, the base may be chosen from strong bases, in particular chosen from the group consisting of NaOH and KOH. According to a particular embodiment, the base is chosen from NaOH, KOH, CaO, Ca(OH)₂ or inorganic carbonates. The base may be added in solid form or as a solution, preferably an aqueous solution. In general, the base concentration is between 0.5N and 5N, preferably between 1N and 4N, very preferentially between 2N and 3N. In cases where the base is introduced in solid form, the biomass is generally used in wet form. Without wishing to be bound by any theory, the moisture contained in the biomass allows the base used to be dissolved.

In general, the biomass and the base are added separately to the extruder. Optionally, a stream of water may also be added to the extruder.

In general, the mass ratio between biomass and base is between 10% and 90%, preferably between 25% and 75%, even more preferentially between 30% and 60%. By way of example, when the aqueous base solution has a concentration of 2N, step (a) may be performed with a mass ratio between biomass and aqueous solution of 50%.

In general, the mass ratio (kg/kg) between the amount of base used and the amount of biomass relative to dry biomass is between 0.05 and 0.50, preferably between 0.05 and 0.30, very preferentially between 0.09 and 0.20. For the calculation of this mass ratio, the amount of base used refers to the mass of base used directly in solid form, or to the mass of solid base present in the base solution used in step a). However, these values may be adjusted as a function of the biomass used, or its moisture content notably.

Advantageously, the yield of ferulic acid and/or salts thereof in step a) is quantitative. The reactive extrusion of step a) generally allows complete release of the ferulic acid contained in the biomass, or of substantially all the ferulic acid contained in the biomass.

Advantageously, the yield of ferulic acid and/or salts thereof in step a) is greater than or equal to 50%, preferably greater than or equal to 60%.

Preferentially, step a) is performed in the absence of hydrolysis enzymes such as cellulases or hemicellulases.

According to another embodiment, the base used in step a) is replaced with at least one enzyme promoting the extraction of ferulic acid and/or salts thereof from the biomass. Without wishing to be bound by theory, the combination of extrusion with the extraction of ferulic acid and/or salts thereof in the presence of a base allows hydrolysis of the cell walls and release of the ferulic acid in salified form in a single unitary operation.

The process of the present invention notably allows the size of the reactor used to be reduced, and also allows the process to be intensified, i.e. a greater amount of biomass can be processed in a certain reactor volume compared to known processes, or a greater amount of biomass can be processed in a certain time compared to known processes, the residence time of the biomass in the extruder being shorter. Advantageously, the environmental impact of the process of the present invention is improved, notably in that the water and/or energy consumption is reduced while at the same time maintaining a good ferulic acid extraction yield.

### Step b)

The raw extrudate (EB) may be subjected to at least one washing step b). Preferably, step b) is performed by adding a solvent, preferably chosen from water, alcohols or a mixture thereof. The addition of solvent is performed so as to suspend the extrudate. Preferably, the water is distilled or demineralized water. The alcohol is generally chosen from alcohols comprising between 1 and 6 carbon atoms, such as methanol, ethanol or isopropanol, preferably ethanol. Advantageously, the use of ethanol allows improved solid/liquid separation, as ferulic acid and/or salts thereof are preferably recovered in the liquid phase. The use of ethanol also allows improved recovery of fibers in the solid phase and/or reduces the amount of dry matter in the liquid phase. Advantageously, the use of ethanol in the washing solvent makes it possible to improve the polysaccharide separation: the polysaccharide content is reduced in the liquid phase. The solvent may also be a water/alcohol mixture; preferably, the water/ethanol mass ratio is generally between 1:1 and 1:3. The solvent may also comprise an acid, preferably chosen from hydrochloric acid, or sulfuric acid.

In general, the suspension is stirred. Step b) is generally performed at a temperature of between 10° C. and 80° C., preferably between 10° C. and 50° C. Preferentially, step b) is generally performed at a temperature of between 10° C. and 35° C. The solid and liquid phases of the suspension obtained in step b) are then separated to allow the preparation of a washed extrudate (EL) and a liquid solution (S).

In general, the pH of the liquid solution (S) is greater than or equal to 5.

According to one embodiment, the pH of the solution (S) is preferably between 5 and 8, preferably between 5 and 6. The liquid solution (S) comprises ferulic acid in salified, dissolved form.

Preferably, the separation may be performed via any process for separating a solid/liquid composition, notably such as filtration, decantation, centrifugation, pressing (filter press, screw press) or flotation. According to a particular aspect, the centrifugation is performed at a speed greater than or equal to 1000 rpm, and preferably less than or equal to 5000 rpm.

According to a particular embodiment of step b), a liquid solution (S1) is isolated from a solid phase, the washed extrudate (EL). Optionally, the solid phase obtained on conclusion of step b) (EL) may be again subjected to step b) to obtain a second liquid solution (S2). This washing may be repeated several times. The subsequent washings may be performed by adding a solvent as described previously, in particular a solvent chosen from water, alcohols or mixtures thereof. Alternatively, the subsequent washings may be performed by recycling the liquid solutions obtained on conclusion of step c) of a process according to the present invention. On conclusion of these repetitions, the liquid solutions (Si) (in which i represents the number of washes performed) are combined to form a liquid solution (S).

In general, the total washing ratio is between 1 and 5, preferably between 1 and 4, very preferentially between 1 and 3 and even more preferentially between 1 and 2.5. The total washing ratio is defined as the ratio between the total amount of liquid used in step (b) relative to the amount of raw extrudate (EB) as defined previously.

In general, the concentration of ferulic acid or salts thereof in the liquid solution (S) is between 0.1 g·L⁻¹ and 30 g·L⁻¹, preferably between 0.5 g·L⁻¹ and 15 g·L⁻¹.

Advantageously, the washed extrudate can be upgraded, notably for anaerobic digestion, animal nutrition or human food.

In general, the amount of dry matter present in the washed extrudate is less than or equal to 70%, preferably less than or equal to 60%, 50%, 40%, 30%, 20%, 10%. The amount of dry matter represents the amount of solid (usually fiber) remaining after complete drying of the washed extrudate.

In general, the amount of dry matter present in the liquid solution (S) is less than or equal to 20%, preferably less than or equal to 10%, very preferentially less than or equal to 5%. According to a particular embodiment, extrusion step a) and washing step b), as described previously, may be performed simultaneously, allowing the preparation of a washed extrudate and a filtrate (F). In the context of this embodiment, the notion of performing steps a) and b) “simultaneously” refers to a case in which all these steps are performed in at least one extruder, for example two consecutive extruders. In general, in the context of this embodiment, the extrusion and washing steps are performed consecutively. In particular, this combination may notably be performed in the presence of an extruder and at least one filter barrel. According to a particular embodiment, the washing may be performed counter-currentwise. In general, the concentration of ferulic acid in the filtrate (F) is between 0.1 g·L⁻¹ and 10 g·L⁻¹. Advantageously, the washed extrudate can be upgraded, notably for anaerobic digestion, animal nutrition or human food.

In general, the amount of dry matter present in the washed extrudate is less than or equal to 70%, preferably less than or equal to 60%, 50%, 40%, 30%, 20%, 10%. The amount of dry matter represents the amount of solid (usually fiber) remaining after complete drying of the washed extrudate.

In general, the amount of dry matter present in the filtrate (F) is less than or equal to 20%, preferably less than or equal to 10%, very preferentially less than or equal to 5%.

### Step c)

The process for extracting ferulic acid and/or salts thereof may comprise at least one step c) of purifying the filtrate (F) and/or the liquid solution (S).

According to a particular aspect, the purification may notably comprise a step c1) of acidifying the filtrate (F) or the liquid solution (S). Step c1) is optional. On conclusion of step c1), an acidified solution is recovered in which ferulic acid is present in protonated form.

The acidification step is generally performed by adding a strong acid, in particular chosen from the group consisting of sulfuric acid, hydrochloric acid and phosphoric acid. In general, on conclusion of the acidification step, the pH of the solution is between 2 and 5, preferably between 2.5 and 4. Advantageously, the acidification step allows precipitation of the fibers and fatty acids contained in the filtrate (F) or in the liquid solution (S).

In general, a filtration step may be performed on conclusion of the acidification step c1), this step notably allowing filtration of the salts obtained during the acidification. Advantageously, filtration also allows the fatty acids to be at least partially retained.

According to another particular aspect, the filtrate (F) or liquid solution (S) may be used directly. In general, the pH of the filtrate (F) or liquid solution (S) is greater than or equal to 5. According to a particular aspect, the pH of the filtrate (F) or liquid solution (S) is generally between 10 and 13, preferably between 11 and 12. According to another embodiment, the pH of the filtrate (F) or solution (S) is preferably between 5 and 8, more preferably between 5 and 6. The liquid solution (S) comprises ferulic acid in salified, dissolved form.

The purification process may also comprise at least one step c2) of adsorption of ferulic acid and/or salts thereof. The adsorption may generally be performed on a column filled with a synthetic resin preferably chosen from the group consisting of Amberlite XAD-4, Amberlite XAD-16, PVDPP (polyvinyl polypyrrolidone), DVBS (divinylbenzene styrene), DVBPS (divinylbenzene polystyrene resins), preferably polyvinyl benzyl dimethyl amine, polyvinyl benzyl dimethyl amine. Alternatively, the adsorption step may be performed on activated charcoal. Step c2) may also be performed with Acticarbone BGX, Acticarbone BGE, Amberlite IRA-900, Ambersep 90OH or Purolite MN502.

On conclusion of the resin adsorption step, a resin or activated charcoal containing ferulic acid and/or salts thereof and an aqueous solution with a reduced content of ferulic acid and/or salts thereof are obtained. Preferably, the aqueous solution has a content of ferulic acid and/or salts thereof of less than or equal to 0.1 g/L. This aqueous solution may advantageously be recycled into the process for extracting ferulic acid and/or salts thereof according to the present invention. In particular, this aqueous solution may be used as a washing solvent in step (b). In general, step c2) allows the polysaccharides to be separated from ferulic acid. Ferulic acid or salts thereof are adsorbed during the adsorption step.

On conclusion of the adsorption step, a step c3) of desorption of ferulic acid and/or salts thereof with a solvent is generally performed. In general, desorption is performed with a counter-current flow. A ferulic acid-rich solution is obtained on conclusion of step c3). Preferably, the ferulic acid-rich solution has a ferulic acid content of between 5 g·L⁻¹ and 500 g·L⁻¹.

Preferably, the solvent used in the desorption step is chosen from the group consisting of water (acidic or basic, with or without complexing agent), cyclic and acyclic hydrocarbon solvents, alcohols, aromatic alcohols, aldehydes, ketones and esters; preferably, the solvent is an alcohol and very preferentially the alcohol is ethanol. In particular, when the solvent used is water, said water has an acidic or basic pH, and may also contain one or more complexing agents. The term “complexing agent” means a substance that is capable of generating a precipitate which is insoluble in the solvent of the liquid medium, in particular insoluble in water. According to one embodiment, the first complexing agent is a cation, in solution in a solvent, preferably in water or in a solvent mixture.

For example, the first complexing agent is advantageously in the form of a monovalent, divalent, trivalent, tetravalent or pentavalent cation salt solution, in particular a divalent or trivalent cation salt.

The cation salt, in particular divalent or trivalent, may be a sulfate, chloride, nitrate, carbonate, phosphate, hydroxide or acetate salt or a mixture thereof.

The cation, notably divalent or trivalent, may be chosen from the group consisting of transition metals, metals, alkaline-earth metals or rare-earth metals, it being understood that the cation, when placed in contact with the starting medium, is capable of forming a precipitate that is insoluble in the solvent of the starting medium, notably in water.

In one embodiment, the first complexing agent is a cation of a transition metal chosen from the group consisting of iron, nickel, copper, titanium, zirconium or a mixture thereof, preferably chosen from iron or copper.

According to one embodiment, the first complexing agent is a metal cation chosen from the group consisting of aluminum and zinc.

According to another embodiment, the first complexing agent is a cation of an alkaline-earth metal chosen from the group consisting of calcium and magnesium.

According to a variant, the first complexing agent is chosen from the group consisting of rare-earth metals such as yttrium or lanthanides, or metal oxides such as Al₂O₃, TiO₂, SiO₂ and/or ZnO.

On conclusion of the desorption step, a liquid fraction rich in ferulic acid and/or salts thereof is obtained. The purification process may comprise an optional step c4) of solvent evaporation to allow the production of purified ferulic acid (FAP). An optional acidification step may be required prior to evaporation to obtain ferulic acid in protonated form.

According to a particular aspect, FAP may be used directly, notably in a process for preparing vanillin by fermentation.

Advantageously, the resin or active charcoal used in step c2) may be regenerated.

According to a particular aspect, the extraction process may also comprise a step in which ferulic acid is crystallized or precipitated.

According to a particular aspect, crystallized or precipitated ferulic acid (FAC) may be used directly, notably in a process for preparing vanillin by fermentation.

According to one embodiment, the purification process may be preceded by a step c0) preliminary to steps c1), c2) and/or c3) in which the alcohol or alcohol mixture, optionally used in the washing step b), is evaporated from the liquid solution (S) or filtrate (F). Preferably, this preliminary step is performed by evaporation, in particular using a falling-film or scraped evaporator.

Advantageously, on conclusion of at least one purification step c) and preferentially on conclusion of steps c0), c1), c2) and/or c3), the amount of residual solvent is less than or equal to 2% by weight, preferably less than or equal to 1% by weight, very preferentially less than or equal to 0.5% and even more preferentially less than or equal to 1000 ppm relative to the dry biomass.

Another aspect of the present invention relates to a process for preparing vanillin by fermentation of ferulic acid and/or salts thereof obtained according to the extraction process of the present invention. In general, the vanillin preparation process is performed in the presence of a microorganism, for example as described in EP0885968 in particular in the presence of Amycolatopsis ATCC 39116.

The ferulic acid or salts thereof that may be used for the preparation of vanillin may be FAP, FAC.

Advantageously, the process of the present invention has improved properties such as:
Reduced environmental footprint, by reducing the water and/or energy consumption,
Reduced size of equipment required for industrialization,
Does not require the use of enzymes,
Simplified separation of polysaccharides and ferulic acid,
The biomass extracted on conclusion of the process according to the present invention may be reused in anaerobic digestion or animal feed,
The other constituents of the biomass are not degraded by the process of the present invention, and the biomass extracted on conclusion of the process according to the present invention may be used to extract other constituents.

The present invention also relates to ferulic acid in protonated or salified form that may be obtained according to the process of the present invention.

The invention finds applications notably in the field of foodstuffs, cosmetics and flavorings.

### EXAMPLES

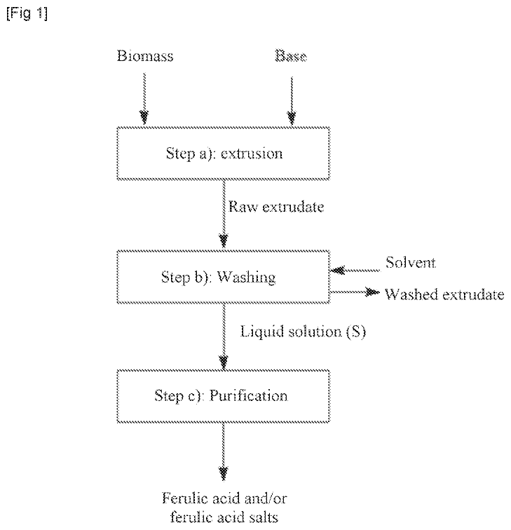

### Example 1: Extraction of Ferulic Acid from Corn Bran (According to FIG. 1)

### Step a): The Extrusion Conditions are Collated in Table 1 Below.

TABLE 1

| Extruder model | Leistritz ZSE 18 60D | |
|---|---|---|
| Base | NaOH (2N) | |
| Ratio | 50% by weight of biomass/base | |
| NaOH/biomass mass ratio (kg/kg) | 0.09 | |
| (relative to the dry base) | | |
| Total flow rate | 2 | kg/h |
| Rotation speed | 200 revolutions per minute (rpm) | |
| Temperature | 60° | C. |

| | |
|---|---|
| Typical profile | 1 feed zone |
| | 3 blending zones |
| | Outlet via convergent |

### Step b): Raw Extrudate Washing and Solid-Liquid Separation

The extrudate obtained in step a) (raw extrudate) is dissolved in water in a standard perfectly stirred reactor (SPSR) at a dilution rate of 10 for 3 h with stirring at room temperature. In this example, the washing ratio is 9.

The solution is separated from the solid phase (washed extrudate) by centrifugal decantation at room temperature. Two streams are thus obtained: the solid with a dry matter (DM) content of 15% and a liquid solution (S) with a DM content of 3%.

The degree of extraction of the ferulic acid contained in the liquid solution (S), measured by HPLC, is equivalent to at least 80% of the ferulic acid content present in the corn bran.

### Step c1): Acidification/Filtration of the Solution

The resulting solution is acidified by adding sulfuric acid to pH 2-3, so as to promote the subsequent adsorption of ferulic acid. The solution is clarified by filtration on a cartridge or centrifugation of the solution before proceeding to the adsorption step.

Step c2), c3): Isolation/Purification of Ferulic Acid

The ferulic acid present in the acidified solution obtained in step c1) is adsorbed on XAD 16-type polystyrene divinylbenzene adsorbent resin. Desorption is performed with ethyl acetate.

Crystallization is performed to obtain ferulic acid in a purity of greater than 80%.

### Example 2: Extraction of Ferulic Acid from Oat Hull

### Step a): The Extrusion Conditions are Collated in the Table Below.

TABLE 2

| Extruder model | Leistritz ZSE 18 60D | |
|---|---|---|
| Base | NaOH (2N) | |
| Ratio | 50% by weight of biomass/base | |
| NaOH/biomass mass ratio | 0.09 | |
| (relative to the dry base) | | |
| Total flow rate | 2 | kg/h |
| Rotation speed | 200 revolutions per minute (rpm) | |
| Temperature | 60° | C. |

| | |
|---|---|
| Typical profile | 1 feed zone |
| | 3 blending zones |
| | Outlet via convergent |

### Step b): Extrudate Repulping in Aqueous Solution

The extrudate obtained in step a) (raw extrudate) is dissolved in water in a standard perfectly stirred reactor (SPSR) at a dilution rate of 10 (washing ratio=9) for 3 h with stirring at room temperature.

The solution is separated from the solid phase (washed extrudate) by centrifugation or dewatering to allow a washed extrudate and a liquid solution (S) to be obtained.

The degree of extraction of the ferulic acid contained in the liquid solution (S) measured by HPLC is equivalent to at least 70% of the ferulic acid content present in the oat hull.

### Example 3: Extraction of Ferulic Acid from Corn Bran

### Step a): The Extrusion Conditions are Collated in the Table Below.

TABLE 3

| Extruder model | Clextral Evolum 32 40D | |
|---|---|---|
| Base | NaOH (2N) | |
| Ratio | 50% by weight of biomass/base | |
| NaOH/biomass mass ratio | 0.09 | |
| (relative to the dry base) | | |
| Total flow rate | 20 | kg/h |
| Rotation speed | 200 revolutions per minute (rpm) | |
| Temperature | 60° | C. |

| | |
|---|---|
| Typical profile | 1 feed zone |
| | 3 blending zones |
| | Outlet without die |

### Step b): Extrudate Repulping in Aqueous-Ethanolic Solution

The extrudate obtained in step a) (raw extrudate) is dissolved in an aqueous-ethanolic solution with a water/ethanol mass ratio of 2:3 in a standard perfectly stirred reactor (SPSR) at a dilution rate of 6 (washing ratio=5) for 3 h with stirring at room temperature.

The solution obtained from step b) is separated from the washed extrudate by centrifugal decantation at room temperature. Two streams are thus obtained: the solid with a dry matter (DM) content of 50% and the liquid solution (S) with a DM content of 3-4%.

The degree of extraction of the ferulic acid contained in the liquid solution (S), measured by HPLC, is equivalent to at least 80% of the ferulic acid content present in the corn bran.

### Example 4: Extraction of Ferulic Acid from Corn Bran (According to FIG. 1)

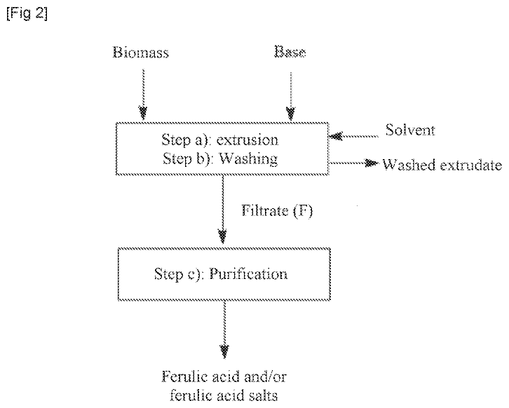

Step a): The extrusion conditions are collated in the table below. A step of washing with water is added during the extrusion profile. The filtration may also be performed directly in the extruder by means of a filter barrel (as shown in FIG. 2).

TABLE 4

| Extruder model | Clextral Evolum 32 40D | |
|---|---|---|
| Base | NaOH (2N) | |
| Ratio | 50% by weight of biomass/base | |
| NaOH/biomass mass ratio | 0.09 | |
| (relative to the dry base) | | |
| Total flow rate | 20 | kg/h |
| Rotation speed | 200 revolutions per minute (rpm) | |
| Temperature | 60° | C. |

| | |
|---|---|
| Typical profile | 1 feed zone |
| | 3 blending zones |
| | 1 washing/neutralization zone |
| | 1 optional filter barrel |
| | 1 counter-thread zone |
| | Outlet without die |

### Step b): Extrudate Repulping in Aqueous-Ethanolic Solution

The extrudate obtained in step 1 is dissolved in an aqueous-ethanolic solution with a water/ethanol mass ratio of 2:3 in a standard perfectly stirred reactor (SPSR) at a dilution rate of 6 (washing ratio=5) for 3 h with stirring at room temperature.

The solution obtained from step b) is separated by centrifugal decantation at room temperature. Two streams are thus obtained: the solid with a dry matter (DM) content of 50% and the liquid solution (S) with a DM content of 3-4%.

The degree of extraction of the ferulic acid contained in the liquid solution (S), measured by HPLC, is equivalent to at least 80% of the ferulic acid content present in the corn bran.

### Step c1): Acidification/Filtration of the Solution

The resulting solution is acidified by adding sulfuric acid to pH 2-3, so as to promote the subsequent adsorption of ferulic acid. The solution is clarified by filtration on a cartridge or centrifugation of the solution before proceeding to the adsorption step.

Step c2), c3): Isolation/Purification of Ferulic Acid

After a step of evaporating off the ethanol, the ferulic acid present in the acidified solution obtained in step c1) is adsorbed on XAD 16-type polystyrene divinylbenzene adsorbent resin. Desorption is performed with ethyl acetate.

Crystallization is performed to obtain ferulic acid in a purity of greater than 80%.

### Example 5: Extraction of Ferulic Acid from Corn Bran

### Equipment Used:

TABLE 5

| | |
|---|---|
| Extruder model | Clextral Evolum 32 40D |
| Extrusion and washing profile | 1 feed zone |
| | 3 blending zones |
| | 1 washing zone composed of an injection |
| | zone and a blending zone |
| | 1 filter barrel |
| | 1 counter-thread zone |
| | Outlet without die |
| Total flow rate | 18 kg/h |

### Operating Conditions of Steps a) and b):

TABLE 6

| Example number | 5a | 5b | 5c | 5d | 5e | 5f | 5g | 5h | 5i | 5j | 5k |
|---|---|---|---|---|---|---|---|---|---|---|---|
| Base | NaOH (2.16N) | | | | | | | | | | |
| NaOH/biomass | 0.089 | | | | | | | | | | |
| mass ratio | | | | | | | | | | | |
| (relative to | | | | | | | | | | | |
| the dry base) | | | | | | | | | | | |
| Temperature | 60 | | | | 15 | | | | | | |
| (° C.) | | | | | | | | | | | |
| Washing solvent | Water | | Water* | | Water | | Water/EtOH** | | | Water/EtOH*** | Water/EtOH**** |
| Rotation speed | 200 | 200 | 200 | 200 | 200 | 200 | 200 | 500 | 200 | 200 | 200 |
| Washing ratio | 1 | 2 | 1 | 2 | 1 | 1.5 | 1.5 | 1.5 | 1 | 1.5 | 1.5 |

*The water used in Examples 5c and 5d is recycled on conclusion of the purification step c).
**Water/Ethanol ratio = 40/60 by weight
***Water/Ethanol ratio = 50/50 by weight
****Water/Ethanol ratio = 60/40 by weight

### Results:

TABLE 7

| Example number | 5a | 5b | 5c | 5d | 5e | 5f | 5g | 5h | 5i | 5j | 5k |
|---|---|---|---|---|---|---|---|---|---|---|---|
| Distribution of | 61.8% | 59.5% | 57.6% | 53.2% | 59.4% | 65.4% | 76.5% | 73.9% | 88.3% | 78.3% | 78.8% |
| the DM in the | | | | | | | | | | | |
| solid at outlet | | | | | | | | | | | |
| Dry matter (DM) | 40.9% | 38.4% | 41.6% | 38.6% | 41.8% | 42.5% | 53.0% | 48.5% | 51.1% | 50.8% | 46.18% |
| content in the | | | | | | | | | | | |
| solid | | | | | | | | | | | |
| Dry matter (DM) | 17.7% | 9.5% | 21.8% | 12.8% | 17.6% | 9.4% | 6.9% | 8.2% | 4.9% | 6.6% | 6.8% |
| content in the | | | | | | | | | | | |
| filtrate (F) | | | | | | | | | | | |
| Yield of step a) | 97.9% | 87.5% | 94.2% | 90.5% | 77.0% | 78.0% | 85.6% | 86.3% | 99.3% | 87.9% | 98.4% |
| Residence time | 50-100 | NM* | NM* | NM* | NM* | NM* | NM* | NM* | NM* | NM* | NM* |
| (seconds) | | | | | | | | | | | |

NM* = not measured

### Example 6: Extraction of Ferulic Acid from Corn Gluten Feed (CGF)

### Step a): The Extrusion Conditions are Collated in Table 8 Below.

TABLE 8

| Extruder model | Clextral Evolum 32 40D | |
|---|---|---|
| Extrusion and washing | 1 feed zone | |
| profile | 3 blending zones | |
| | 1 washing zone composed of an injection | |
| | zone and a blending zone | |
| | 1 filter barrel | |
| | 1 counter-thread zone | |
| | Outlet without die | |
| Base | NaOH (2N) | |
| Ratio | 50% by weight of biomass/base | |
| NaOH/biomass mass ratio | 0.09 | |
| (relative to the dry base) | | |
| Total flow rate | 18 | kg/h |
| Rotation speed | 200 revolutions per minute (rpm) | |
| Temperature | 60° | C. |
| Temperature of | 60° | C. |
| the filter barrel | | |

| | |
|---|---|
| Washing solvent | Water/Ethanol (4.5% by mass)/sulfuric acid |
| | (4.1% by mass) |
| Washing ratio | 1.2 |

Two streams are thus obtained: the solid with a dry matter (DM) content of 40% and a liquid solution (S) with a DM content of 18-19%.

The pH of the extracted fiber is 6, and it may advantageously be upgraded in animal feed.

### Example 7: Extraction of Ferulic Acid from Corn Fiber

### Steps a) and b): the Extrusion and Washing Conditions are Collated in the Table Below.

TABLE 9

| Extruder model | Clextral Evolum 32 40D | |
|---|---|---|
| Extrusion and washing | 1 feed zone | |
| profile | 3 blending zones | |
| | 1 washing zone composed of an injection | |
| | zone and a blending zone | |
| | 1 filter barrel | |
| | 1 counter-thread zone | |
| | Outlet without die | |
| Total flow rate | 18 | kg/h |
| Rotation speed | 200 revolutions per minute (rpm) | |
| Base | NaOH (2N) | |
| Ratio | 50% by weight of biomass/base | |
| NaOH/biomass mass ratio | 0.09 | |
| (relative to the dry base) | | |
| Rotation speed | 200 revolutions per minute (rpm) | |
| Temperature | 60° | C. |
| Temperature of | 60° | C. |
| the filter barrel | | |

| | |
|---|---|
| Washing solvent | Water/Ethanol (4.5% by mass)/sulfuric acid |
| | (4.1% by mass) |
| Washing ratio | 1.2 |

Two streams are thus obtained: the solid with a dry matter (DM) content of 41% and a liquid solution (S) with a DM content of 18-19%.

The pH of the extracted fiber is 6, and it may advantageously be upgraded in animal feed.

### Example 8—Extraction of Ferulic Acid from Corn Gluten Feed

### Step a): The Extrusion Conditions are Collated in Tables 10 and 11 Below.

TABLE 10

| | Example number | | | | |
|---|---|---|---|---|---|
| | 6a | 6b | 6c | 6d | 6e |
| Extruder | Clextral Evolum 32 40D | | | | |
| model | | | | | |
| Extrusion | 1 feed zone | | | | |
| profile | 3 blending zones | | | | |
| Base | NaOH (1.8N) | | | | |
| Biomass/base | 50 | 60 | 40 | 40 | 60 |
| mass | | | | | |
| ratio (%) | | | | | |
| NaOH/biomass | 0.076 | 0.051 | 0.115 | 0.114 | 0.051 |
| mass ratio | | | | | |
| (relative to | | | | | |
| the dry base) | | | | | |
| Total flow | 12 | 15 | 13.3 | 16.7 | 25 |
| rate | | | | | |
| (kg/hour) | | | | | |
| Rotation | 100 | 500 | 500 | 300 | 100 |
| speed | | | | | |
| revolutions | | | | | |
| per minute | | | | | |
| (rpm) | | | | | |

| | |
|---|---|
| Temperature | 60° C. |

TABLE 11

| Example number | 6f | 6g | 6h | 6i | 6j | 6k | 6l | 6m |
|---|---|---|---|---|---|---|---|---|
| Extruder model | Clextral Evolum 32 40D | | | | | | | |
| Extrusion profile | 1 feed zone | | | | | | | |
| | 3 blending zones | | | | | | | |
| Base | NaOH (2.2N) | | | | | NaOH (2N) | | |
| Biomass/base | 60 | 40 | 60 | 50 | 40 | 40 | 50 | 60 |
| mass ratio (%) | | | | | | | | |
| NaOH/biomass | 0.062 | 0.139 | 0.062 | 0.093 | 0.138 | 0.127 | 0.084 | 0.056 |
| mass ratio | | | | | | | | |
| (relative to | | | | | | | | |
| the dry base) | | | | | | | | |
| Total flow rate | 15 | 10 | 20 | 20 | 16.7 | 10 | 16 | 25 |
| (kg/hour) | | | | | | | | |
| Rotation speed | 300 | 500 | 100 | 500 | 100 | 100 | 300 | 500 |
| revolutions per | | | | | | | | |
| minute (rpm) | | | | | | | | |

| | |
|---|---|
| Temperature | 60° C. |

### Results:

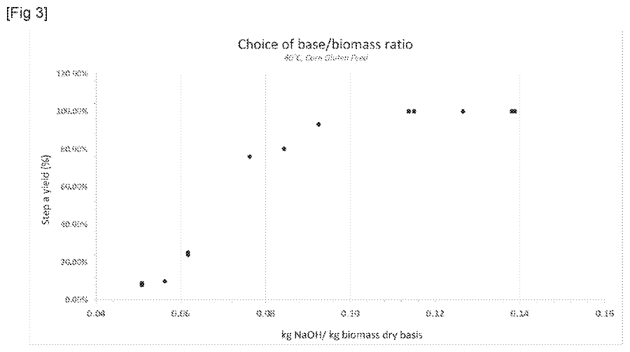

The results show that the only factor affecting the yield of the extrusion step a) is the ratio of base to biomass (see Table 12 and FIG. 3).

TABLE 12

| Example number | 6a | 6b | 6c | 6d | 6e | 6f | 6g | 6h | 6i | 6j | 6k | 6l | 6m |
|---|---|---|---|---|---|---|---|---|---|---|---|---|---|
| Yield of step a) (%) | 76 | 9 | 100 | 100 | 8 | 24 | 100 | 25 | 93 | 100 | 100 | 80 | 10 |

### Example 9: Extraction of Ferulic Acid from Corn Bran

### Step a): The Extrusion Conditions are Collated in the Table Below.

TABLE 13

| Extruder model | Clextral Evolum 32 40D | |
|---|---|---|
| Base | NaOH (2N) | |
| Ratio | 50% by weight of biomass/base | |
| NaOH/biomass mass ratio | 0.09 | |
| (relative to the dry base) | | |
| Total flow rate | 20 | kg/h |
| Rotation speed | 200 revolutions per minute (rpm) | |
| Temperature | 60° | C. |

| | |
|---|---|
| Typical profile | 1 feed zone |
| | 3 blending zones |
| | Outlet without die |

### Step b): Repulping of 150 kg of Extrudate in Aqueous-Ethanolic Solution

The extrudate obtained in step a) (raw extrudate) is dissolved in an aqueous-ethanolic solution with a water/ethanol mass ratio of 2:3 in a perfectly stirred reactor at a dilution rate of 6 (washing ratio=5) for 2 h with stirring at room temperature.

The solution obtained from step b) is separated from the washed extrudate by centrifugal decantation at room temperature. Two streams are thus obtained: the solid (EL) with a dry matter (DM) content of 52% and the liquid solution (S) with a DM content of 1.5%.

The degree of extraction of the ferulic acid contained in the liquid solution (S), measured by HPLC, is 77% of the ferulic acid content present in the corn bran.

The fiber yield obtained in the solid (E) is 94% of the initial content of biomass introduced. According to the analyses of composition and nutritional properties measured on the solid (E), the milk fodder unit (UFL) is 1.05 on a dry basis.

The methane formation potential is measured at about 170 Nm³/t (cubic normometer per ton) on a dry basis.

step c): The liquid solution (S) is concentrated in a falling-film evaporator to remove the ethanol, followed by acidification to pH 2 with sulfuric acid. A centrifugation and clarification step is performed on the solution (Sbis), concomitantly allowing the fatty acids present in the solution to be removed.

### Example 10: Extraction of Ferulic Acid from Corn Bran

### Steps a) and b): The Extrusion and Washing Conditions are Collated in Table 14 Below.

TABLE 14

| Extruder model | Clextral Evolum 32 40D | |
|---|---|---|
| Extrusion and washing profile | 1 feed zone | |
| | 3 blending zones | |
| | 1 washing zone composed of an injection | |
| | zone and a blending zone | |
| | 1 filter barrel | |
| | 1 counter-thread zone | |
| | Outlet without die | |
| Total flow rate | 18 | kg/h |
| Rotation speed | 200 revolutions per minute (rpm) | |
| Base | NaOH (2N) | |
| Ratio | 50% by weight of biomass/base | |
| NaOH/biomass mass ratio | 0.09 | |
| (relative to the dry base) | | |
| Temperature | 60° | C. |
| Temperature of the | 60° | C. |
| filter barrel | | |

| | |
|---|---|
| Washing solvent | Water |
| Washing ratio | 2 |

Two streams are thus obtained: the solid with a dry matter (DM) content of 38% and a filtrate (F) with a DM content of 8%.

The pH of the solid (extracted fiber) is 12, and it may advantageously be upgraded directly in anaerobic digestion.

### Example 11; Extraction of Ferulic Acid from Corn Bran

### Steps a) and b): The Extrusion and Washing Conditions are Collated in the Table Below.

TABLE 15

| Extruder model | Clextral Evolum 32 40D | |
|---|---|---|
| Extrusion and washing profile | 1 feed zone | |
| | 3 blending zones | |
| | 1 washing zone composed of an injection | |
| | zone and a blending zone | |
| | 1 filter barrel | |
| | 1 counter-thread zone | |
| | Outlet without die | |
| Total flow rate | 18 | kg/h |
| Base | NaOH (2N) | |
| Ratio | 50% by weight of biomass/base | |
| NaOH/biomass mass ratio | 0.09 | |
| (relative to the dry base) | | |
| Rotation speed | 200 revolutions per minute (rpm) | |
| Temperature | 60° | C. |
| Temperature of the | 60° | C. |
| filter barrel | | |

| | |
|---|---|
| Washing solvent | Water/sulfuric acid (70/30) |
| Washing ratio | 2 |

Two streams are thus obtained: the solid with a dry matter (DM) content of 39% and a filtrate (F) with a DM content of 10%.

The mean residence time is 50 seconds.

The pH of the solid (extracted fiber) is 5-6, and it may advantageously be upgraded directly in anaerobic digestion or directly in animal feed.

### Example 12: Extraction of Ferulic Acid from CGF

### Steps a) and b): The Extrusion and Washing Conditions are Collated in the Table Below.

TABLE 16

| Extruder model | Clextral Evolum 53 48D | | |
|---|---|---|---|
| Extrusion and | 1 feed zone | | |
| washing profile | 3 blending zones | | |
| | 1 washing zone composed of an injection zone and a blending zone | | |
| | 1 filter barrel | | |
| | 1 counter-thread zone | | |
| | Outlet without die | | |
| Total flow rate | 120 kg/h | 300 kg/h | 600 kg/h |
| Rotation speed | 450 revolutions | 600 revolutions | 1200 revolutions |
| | per minute | per minute | per minute |
| | (rpm) | (rpm) | (rpm) |
| Base | NaOH (2N) | | |
| Ratio | 50% by weight of biomass/base | | |
| Temperature | 60° C. | | |
| Temperature of | 60° C. | | |
| the filter barrel | | | |
| Washing | Water/Ethanol (4.5% by mass)/sulfuric acid (4.1% by mass) | | |
| solvent | | | |
| Washing ratio | 1.2 | | |
| Dry matter (DM) | 39% | 47% | 52% |
| content in the | | | |
| solid | | | |
| Dry matter (DM) | 15% | 14% | 14% |

content in the
filtrate (F)

The pH of the solid (extracted fiber) is 5-6, and it may advantageously be upgraded as animal feed or in anaerobic digestion.

### Example 13: Extraction of Ferulic Acid from Corn Gluten Fiber

### Steps a) and b): The Extrusion and Washing Conditions are Collated in the Table Below.

TABLE 17

| Extruder model | Clextral Evolum 53 48D | | | |
|---|---|---|---|---|
| Extrusion and | 1 feed zone | | | |
| washing profile | 3 blending zones | | | |
| | 1 washing zone composed of an injection zone and a blending zone | | | |
| | 1 filter barrel | | | |
| | 1 counter-thread zone | | | |
| | Outlet without die | | | |
| Total flow rate | 90 kg/h | 90 kg/h | 90 kg/h | 180 kg/h |
| Rotation speed | 160 revolutions | 160 revolutions | 160 revolutions | 450 revolutions |
| | per minute | per minute | per minute | per minute |
| | (rpm) | (rpm) | (rpm) | (rpm) |
| Base | NaOH (2N) | | | |
| Ratio | 50% by weight of biomass/base | | | |
| NaOH/biomass | 0.09 | | | |
| mass ratio | | | | |
| (relative to the dry | | | | |
| base) | | | | |
| Temperature | 60° C. | | | |
| Temperature of | 60° C. | | | |
| the filter barrel | | | | |
| Number of 0.5D | 2 | 3 | 4 | 4 |
| counter-threads in | | | | |
| the profile | | | | |
| Washing solvent | Water/Ethanol (4.5% by mass)/sulfuric acid (4.1% by mass) | | | |
| Washing ratio | 1.2 | | | |
| Dry matter (DM) | 40% | 43% | 45% | 47% |
| content in the | | | | |
| solid | | | | |
| Dry matter (DM) | 13% | 10% | 14% | 11% |

content in the
filtrate (F)

The pH of the solid (extracted fiber) is 5-6, and it may advantageously be upgraded in anaerobic digestion.

1. A process for preparing ferulic acid and/or salts thereof, the process comprising:
a step a) of extruding a biomass in the presence of a base to prepare a raw extrudate (EB) and optionally a filtrate (F), wherein a mass ratio between an amount of the base and an amount of the biomass relative to a dry biomass is between 0.05 and 0.50.

2. The preparation process according to claim 1, further comprising: at least one step b) of washing the raw extrudate (EB) with a solvent to prepare a washed extrudate (EL) and a liquid solution (S).

3. The preparation process according to claim 1, further comprising: at least one step c) of purifying the filtrate (F) and/or the liquid solution (S).

4. The process according to claim 1, wherein the base is selected from the group consisting of strong bases.

5. The process according to claim 1, wherein the base is selected from NaOH, KOH, CaO, Ca(OH)₂ or inorganic carbonates.

6. The process according to claim 2, wherein a total washing ratio is between 1 and 5.

7. The process according to claim 2, wherein the washed extrudate (EL) and the liquid solution (S) are separated by filtration, decantation, centrifugation, pressing or flotation.

8. The process according to claim 1, further comprising step c) of purifying the filtrate (F) or the liquid solution (S), wherein step c) comprises:
optionally, a step c1) of acidifying the filtrate (F) or the liquid solution (S),
a step c2) of adsorbing ferulic acid,
a step c3) of desorbing ferulic acid and/or salts thereof with a solvent, and
optionally, a step c4) of evaporating off the desorption solvent from step c3), thereby producing a purified ferulic acid (FAP).

9. A process for preparing ferulic acid and/or salts thereof, comprising:
a step a) of extruding a biomass in the presence of a base to prepare a raw extrudate (EB), and
a washing step b) to prepare a washed extrudate (EL) and a liquid solution (S) or a filtrate (F),
wherein the washing step b) is performed by adding a solvent, wherein the solvent is a water/alcohol mixture.

10. The process according to claim 9, wherein a total washing ratio is between 1 and 5.

11. The process according to claim 9 further comprising: at least one step c) of purifying the filtrate (F) and/or the liquid solution (S).

12. The process according to claim 9, wherein the base is selected from the group consisting of strong bases.

13. The process according to claim 9, wherein the base is selected from NaOH, KOH, CaO, quicklime or inorganic carbonates.

14. The process according to claim 9, wherein a mass ratio between an amount of the base used and an amount of the biomass relative to a dry biomass is between 0.05 and 0.50.

15. The process according to claim 9, wherein the washed extrudate (EL) and the liquid solution (S) are separated by filtration, decantation, centrifugation, pressing or flotation.

16. The process according to claim 9, further comprising a step c) of purifying the filtrate (F) or the liquid solution (S), wherein step c) comprises:
optionally, a step c1) of acidifying the filtrate (F) or the liquid solution (S),
a step c2) of adsorbing ferulic acid,
a step c3) of desorbing ferulic acid and/or salts thereof with a solvent, and
optionally, a step c4) of evaporating off the desorption solvent from step c3), thereby producing a purified ferulic acid (FAP).

17. The process according to claim 9, further comprising: a step of crystallization or precipitation of the crystallized ferulic acid (FAC).

18. A process for preparing natural vanillin, comprising converting ferulic acid and/or salts thereof obtained via the process as defined in claim 1, in the presence of a microorganism.

19. A process for preparing natural vanillin, comprising converting ferulic acid and/or salts thereof obtained via the process as defined in claim 9, in the presence of a microorganism.

20. The process according to claim 4, wherein the strong base is selected from the group consisting of NaOH and KOH.
